(19) United States
(12) Patent Application Publication
(10) Pub. No.: US 2023/0167474 A1
(43) Pub. Date: Jun. 1, 2023

(54) Methods for enzymatic production of glucosamine salts and the purification methods thereof

(71) Applicant: JIANGSU HARVERS BIOTECH CO., LTD., Zhenjiang (CN)

(72) Inventors: Chunhua DING, Zhenjiang (CN); Wenjie ZHANG, Zhenjiang (CN)

(21) Appl. No.: 18/078,159

(22) Filed: Dec. 9, 2022

Related U.S. Application Data
(63) Continuation of application No. PCT/CN2020/111623, filed on Aug. 27, 2020.

(30) Foreign Application Priority Data
Jun. 11, 2020 (CN) 2020105278803

(51) Int. Cl. C12P 19/26; C07H 1/06; C07H 5/06

(52) U.S. Cl. CPC C12P 19/26; C07H 1/06; C07H 5/06

(57) ABSTRACT

Disclosed in the present disclosure are methods for enzymatic production of glucosamine salts and the purification methods thereof, and belongs to the technical field of biological engineering. In the present disclosure, liquid containing N-acetylglucosamine is used as a raw material, subjected to hydrolysis with deacetylase to obtain glucosamine and acetic acid, and followed by elution on a cation exchange column with an acidic eluent and separation to obtain the glucosamine salt. Meanwhile, a by-product, namely sodium acetate, is recovered by anion exchange. The obtained glucosamine salt is subjected to concentration, crystallization, decolorization, and drying to obtain a high-purity glucosamine salt crystal. According to the present disclosure, processes for recycling of an enzyme, a residual substrate, and acetic acid are combined. Moreover, the loss rate of resin is low under operation conditions at room temperature, and the production of a hydrochloric acid waste liquid is extremely low.

### TECHNICAL FIELD

The present disclosure relates to methods for enzymatic production of glucosamine salts and the purification methods thereof, and belongs to the technical field of biological engineering.

### BACKGROUND

As an important hexosamine, glucosamine (GlcNAc) is formed by substitution of a hydroxyl of glucose with an amino, and is easily soluble in water and hydrophilic solvents. The glucosamine is widely found in nature, has a chemical name of 2-amino-2-deoxy-D-glucose, and is usually found in polysaccharides and conjugated polysaccharides derived from microorganisms and animals in the form of N-acetyl derivatives (such as chitin) or in the form of N-sulfate and N-acetyl-3-O-lactate ether (muramic acid). A glucosamine hydrochloride with a molecular weight of 215.5 Da is white crystalline, odorless, slightly sweet, easily soluble in water, slightly soluble in methanol and insoluble in ethanol and other organic solvents. A glucosamine molecule is unstable, and is likely to be oxidized or degraded. The glucosamine can be prepared into a glucosamine salt, such as a glucosamine hydrochloride, a glucosamine sulfate, a glucosamine phosphate, and a glucosamine pyruvate, so that the stability can be significantly improved. The glucosamine has important physiological functions on the human body, is involved in detoxification of the liver and kidney to achieve the effects of eliminating inflammation, protecting the liver and replenishing the kidney, has a good curative effect on treatment of rheumatic joint inflammation and gastric ulcer, and is used as a main raw material of synthetic antibiotics and anti-cancer drugs. The glucosamine can also be used in food, cosmetics, and feed additives.

At present, the following three methods are mainly used for production of the GlcNAc: a chemical method, an enzymatic method, and a microbial method. Natural raw materials, such as shrimp and crab shells and fungal cell walls, are rich in chitin, and can be subjected to acid hydrolysis or enzymatic hydrolysis to obtain a glucosamine monomer. The enzymatic method mainly includes specific hydrolysis of the chitin with chitinase, and the enzyme involved mainly includes endochitinase, exochitinase, β-N-hexosaminidase, and deacetylase. A glucosamine monomer can be obtained by enzymatic hydrolysis of the chitin.

With rapid development of genetic engineering, metabolic engineering and synthetic biology, the GlcNAc can be directly biosynthesized by a recombinant microorganism with glucose as a substrate, and the product can even have a concentration of greater than 100 g/L, so that a good foundation is laid for the production of the GlcNAc in a large scale. The method for the production of the glucosamine by microbial fermentation has the advantages of high conversion rate, high product concentration, and short production cycle. However, at present, raw materials for extraction of the glucosamine mainly include the shrimp and crab shells and microbial fermentation liquids. Either a fermentation liquid or an enzymatic hydrolysis product is used, various by-products are produced and incompletely reacted residues are remained while a main product is obtained after a reaction. Therefore, corresponding processes for extraction of the glucosamine are required to be developed according to different raw materials. However, current extraction methods in an actual production process usually have the defects of complicated process route, low separation efficiency, high energy consumption, and heavy environmental pollution.

When a liquid rich in N-acetylglucosamine or a chitin hydrolysate is used as a raw material, the first step during extraction is to remove acetyl in the GluNAc molecule. A method for removing the acetyl mainly includes an acid hydrolysis method and an enzymatic hydrolysis method. According to the acid hydrolysis, a large number of inorganic acids are required to be consumed, and in a subsequent extraction process, a large number of alkaline solutions are required to be added to neutralize the inorganic acid solutions added before, so that a large number of salts are produced in the extraction process. The consumption of acid and alkali is high in the extraction process, and a lot of high-salt wastewater which is difficult to treat is produced. However, according to the enzymatic method for removing the acetyl, a large number of acid and alkaline solutions are not required to be used. Thus, the enzymatic method has received more and more attention.

According to ZL2016112278411 (with a publication number of CN 106831894 B), a method for separating a D-glucosamine hydrochloride by deacetylation coupled adsorption is disclosed. The method includes using an N-acetylglucosamine fermentation liquid as a starting raw material; removing microbial cells by separation with a ceramic membrane; N-acetylglucosamine is obtained by activated carbon decolorization and ion exchange resin method to remove residual salts in the medium; subjecting the N-acetylglucosamine to a deacetylation reaction and adsorption with an acidic cation exchange column at 91° C., where the reaction is carried out for about 120 minutes; and then conducting elution with hydrochloric acid to obtain a glucosamine hydrochloride. Since the reaction temperature is greater than 90° C., a pigment substance is likely to be produced during treatment, and the ion exchange resin is likely to be broken and lost.

According to ZL2013106719979 (with a publication number of CN 103626809 B), a method for purification of a glucosamine hydrochloride mother liquor is disclosed. With the glucosamine hydrochloride mother liquor as a raw material, glucosamine is adsorbed to a positive column by an acidic cation exchange column. After the positive column is subjected to elution with a hydrochloric acid solution, an obtained eluate is treated with an anion exchange column to remove acetic acid, chloride ions, and other anions so as to obtain the glucosamine rather than a glucosamine hydrochloride. Since the glucosamine cannot be stored stably for a long time, the method has a limited application value in industry.

### SUMMARY

In view of the defects of high energy consumption and high pollution in the prior art, the present disclosure provides a novel method for production, separation and purification of a glucosamine salt. A fermentation liquid rich in N-acetylglucosamine or an enzymatic hydrolysate of chitin is used as a raw material. On the basis of obtaining a glucosamine salt by cation exchange, acetate is obtained by anion exchange, and incompletely reacted N-acetylglucosamine is recovered. While the yield of a target product is increased, resource utilization of a by-product is achieved. Moreover, the consumption of raw and auxiliary materials and the emission of wastewater and solid waste are reduced, so that the purposes of energy conservation, consumption reduction, environmental protection, and safety are achieved. According to the method, corresponding glucosamine salts can also be obtained by simply adjusting the type of an acidic eluent for a cation exchange resin, and a variety of high-purity crystals of the glucosamine salts are obtained in an industrial production scale.

A first objective of the present disclosure is to provide a method for production, separation and purification of a glucosamine salt. The method includes the following steps:

(1) using a clear solution containing glucosamine as a raw material, or optionally, using a filtered clear solution containing glucosamine as a raw material after a turbid solution containing glucosamine is filtered with an ultrafiltration membrane, where the ultrafiltration membrane has a molecular weight cut-off of 5-200 kDa;

(2) subjecting the solution containing glucosamine in step (1) to adsorption with a cation exchange resin to make the glucosamine adsorbed to the cation exchange resin;

(3) subjecting the cation exchange resin in step (2) to elution with an acidic eluent to obtain an eluate containing a glucosamine salt;

subjecting a positive column effluent flowing through the cation exchange resin in step (2) to make the acetate ion to be adsorbed on the anion exchange resin; and recycling a negative column effluent containing N-acetylglucosamine flowing through the anion exchange resin to participate in preparation of glucosamine; and

(4) subjecting the anion exchange resin to elution with an alkaline eluent to obtain a eluate containing sodium acetate, where the eluate containing sodium acetate can be used for nitrogen and phosphorus removal processes in a sewage treatment plant, and can also be used as a raw material for chemical reactions and other suitable cases.

In an embodiment, in step (1), the solution containing glucosamine is a reaction product of deacetylation of N-acetylglucosamine by a biological method or a chemical method, and may also be a solution containing glucosamine from another source.

In an embodiment, in step (1), the ultrafiltration membrane may be a membrane assembly prepared from a ceramic material or a membrane assembly prepared from an organic material.

In an embodiment, in step (1), the solution containing glucosamine is prepared by a catalytic reaction with a solution containing N-acetylglucosamine as a raw material and a deacetylase extract or a deacetylase preparation as a catalyst.

In an embodiment, in step (3), the recycling to participate in preparation of glucosamine includes using an enzymatic hydrolysis raw material for preparation of glucosamine in a deacetylation reaction under catalysis of deacetylase.

A second objective of the present disclosure is to provide a method for preparing a glucosamine salt. The method includes subjecting a solution containing N-acetylglucosamine to enzymatic hydrolysis to remove acetyl group first, and then conducting separation and purification according to the separation and purification method.

In an embodiment, the solution containing N-acetylglucosamine may be obtained by microbial fermentation, enzymatic hydrolysis of a biological raw material containing chitin, or chemical hydrolysis of a raw material containing chitin.

In an embodiment, the enzymatic hydrolysis includes using an N-acetylglucosamine solution with a concentration of 40-150 g/L as a raw material, and adding deacetylase in a proportion of 10-40 U/g of N-acetylglucosamine. An enzymatic hydrolysis reaction is carried out under stirring in a pH range of 4-8 at a temperature of 25-55° C. for 10-40 minutes. The N-acetylglucosamine solution is a raw material solution containing N-acetylglucosamine obtained by microbial fermentation or hydrolysis of chitin.

In an embodiment, the deacetylase may be obtained by fermentation of the microorganism, or may be extracted from other organisms. The microorganism may be a microorganism screened in nature or a recombinant microorganism modified through genetic engineering procedures.

In an embodiment, the enzymatic hydrolysis includes carrying out an enzymatic reaction with deacetylase to specifically remove the acetyl in an N-acetylglucosamine molecule, and obtaining an enzymatic hydrolysis product including glucosamine and acetic acid as main components after the reaction.

In an embodiment, the method includes the following steps:

(1) using an N-acetylglucosamine solution with a concentration of 80-150 g/L as a raw material, adding deacetylase in a proportion of 10-40 U/g of N-acetylglucosamine, and carrying out an enzymatic reaction under stirring in a pH range of 4-8 at a temperature of 25-55° C. for 10-90 minutes;

(2) subjecting an enzymatic hydrolysis product obtained after the reaction in step (1) to treatment in step (3) directly or to filtration with an ultrafiltration membrane to obtain an ultrafiltration membrane dialysate containing glucosamine and a membrane concentrate separately, and recycling an enzyme solution of the membrane concentrate to participate in a next enzymatic reaction process in step (1), where the ultrafiltration membrane has a molecular weight cut-off of 5-200 kDa;

(3) subjecting the membrane dialysate containing glucosamine obtained in step (2) to adsorption with a cation exchange resin, and subjecting the cation exchange resin to continuous elution with an acidic eluent to obtain an eluate containing a glucosamine salt;

(4) subjecting a positive column effluent flowing through the cation exchange resin in step (3) to adsorption with an anion exchange resin, and subjecting the anion exchange resin to elution with an alkaline eluent to separate an eluate containing sodium acetate, where the eluate containing sodium acetate can be used for nitrogen and phosphorus removal processes in a sewage treatment plant; and

(5) subjecting a negative column effluent flowing through a negative column in step (4) to filtration with a nanofiltration membrane or a reverse osmosis membrane, and recycling a nanofiltration membrane or reverse osmosis membrane concentrate to participate in a next enzymatic reaction process in step (1).

In an embodiment, in step (3), a device for cation exchange chromatography may be a fixed bed, a continuous bed for ion exchange, or a simulated moving bed for ion exchange. An acidic eluent may be hydrochloric acid, sulfuric acid, phosphoric acid, pyruvic acid, or citric acid. Corresponding glucosamine salts obtained after the elution include a glucosamine hydrochloride, a glucosamine sulfate, a glucosamine phosphate, a glucosamine pyruvate, and a glucosamine citrate respectively. The acidic eluent has a concentration of 0.30-3.0 mol/L.

In an embodiment, in step (3), during the cation exchange, the adsorption and the elution are conducted at a temperature of 20-70° C. In step (4), during the anion exchange, the adsorption and the elution are conducted at a temperature of 20-65° C. The two-level ion exchange chromatography is conducted at a feeding flow rate of 2.0-10.0 BV/h. The eluent has a flow rate of 1.0-8.0 BV/h.

In an embodiment, in step (4), a device for anion exchange chromatography may be a fixed bed, a continuous bed for ion exchange, or a simulated moving bed for ion exchange. An alkaline eluent may be a NaOH solution or a KOH solution. The alkaline eluent has a concentration of 0.30-3.0 mol/L. Sodium acetate or potassium acetate may be separately recovered from the eluate obtained after the elution on the anion exchange column. The sodium acetate or the potassium acetate may be collected through a pipe and transported to a sewage treatment workshop to serve as a supplementary carbon source for nitrogen and phosphorus removal in a sewage treatment process, and may also be used as a raw material for other chemical reaction processes.

In an embodiment, in step (5), the nanofiltration membrane is a ceramic membrane with a pore size of 0.5-2 nm and an operation process of 2-5 atm. The reverse osmosis membrane is an organic spiral-wound membrane or a ceramic membrane with a molecular weight cut-off of 50-100 Da and an operation pressure of 4-10 atm.

In an embodiment, after step (5), concentration, crystallization, and drying are further conducted in sequence.

In an embodiment, the concentration is evaporation concentration. The evaporation concentration may be single-effect evaporation, double-effect evaporation, or multiple-effect evaporation.

In an embodiment, the crystallization is conducted at a temperature of 5-40° C.

In an embodiment, decolorization of a mother liquor is further conducted. A decolorization method is adsorption decolorization with activated carbon. A decolorized mother liquor is recycled in a concentration process. According to the decolorization method, the use amount of the activated carbon is 0.01-2.0% (w/v) of that of a raw material solution.

In an embodiment, the drying is vacuum drying or flash drying. The flash drying is conducted at an inlet air temperature of 110-290° C. and an outlet air temperature of 70-90° C. The low-temperature vacuum drying is conducted at a temperature of 40-80° C. and a vacuum degree of 70-95 kPa.

In an embodiment, the multiple-effect evaporation concentration is triple-effect evaporation, which is conducted at a temperature of 80° C., 70° C., and 60° C. separately. A last-effect evaporator has a vacuum degree of 80-98 kPa.

Beneficial Effects:

Compared with the prior art, the present disclosure has the following advantages.

(1) According to the present disclosure, enzymatic production and efficient purification of different kinds of glucosamine salts are achieved in an industrial production scale. The production process has the advantages of high conversion rate, high recovery rate, low consumption of raw materials, environmental protection, and safety. The product recovery rate is greater than 95%, and the produced glucosamine salt has a purity of greater than 99.5%.

(2) The enzyme used in the present disclosure has mild reaction conditions and high reaction rate. A process for recycling of deacetylase is designed. On the one hand, the enzyme and the product are effectively separated by filtration with an ultrafiltration membrane. On the other hand, the enzyme is recycled, and the use cost of the enzyme is reduced.

(3) In the present disclosure, a recycling process is formed by combining the two-level ion exchange with the reverse osmosis membrane/nanofiltration membrane. On the one hand, the by-product (acetate) and the incompletely reacted substrate (N-acetylglucosamine) are effectively recycled, so that the conversion rate of the substrate and the extraction and recovery rate of the product are effectively increased. On the other hand, the consumption of the resin and the eluent in the ion exchange process is effectively reduced, so that the triple benefits of low consumption, energy conservation, and environmental protection are achieved.

(4) In the present disclosure, the ion exchange process is carried out through a continuous moving bed and a simulated moving bed, so that the separation efficiency during the ion exchange can be improved, and meanwhile, the operation continuity and automation level of the process are improved.

(5) In the present disclosure, the ion exchange is conducted under mild reaction conditions. Compared with a commonly used fixed bed, the consumption of resin can be reduced by about 60%, the consumption of an acidic solution and an alkaline solution required for regeneration of the resin is reduced by about 50%, and meanwhile, the production of wastewater can be greatly reduced.

(6) The process of the present disclosure is suitable for production of a variety of glucosamine salts. The glucosamine salts of corresponding acids can be obtained by changing the type of the acidic eluent for the cation exchange resin. The method has a wide industrial application value.

### BRIEF DESCRIPTION OF FIGURES

FIG. 1 shows an extraction process route of a glucosamine salt.

### DETAILED DESCRIPTION

### Technical Terms

A glucosamine salt refers to a glucosamine salt product obtained after elution on a cation exchange column with different acids, including but not limited to any one of a glucosamine hydrochloride, a glucosamine sulfate, a glucosamine phosphate, a glucosamine pyruvate, and a glucosamine citrate.

A membrane dialysate refers to a liquid flowing through a membrane material during filtration with a membrane.

A membrane concentrate refers to a liquid that cannot flow through a membrane material and is retained during filtration with a membrane.

An eluate refers to a liquid obtained after an eluent (such as an acidic solution or an alkaline solution) flows through a saturated ion exchange resin during ion exchange.

A column effluent refers to a liquid that is not adsorbed by an ion exchange resin, directly flows out from a raw material solution and is washed out by deionized water during ion exchange chromatography.

The activity unit of deacetylase is defined as 1 U=1 mmol/min, and that is to say, 1 mmol of glucosamine obtained after a reaction for 1 minute is defined as 1 U enzyme activity unit.

The purification and recovery rate of a glucosamine salt is as follows: since an enzymatic reaction, ion exchange, and other units are involved in a method for enzymatic production of the glucosamine salt and a purification process therefor, the molecular structure of a reactant is changed. The recovery rate is calculated based on the mass of glucosamine.

The glucosamine salt and N-acetylglucosamine are quantified by an HPLC analysis method. An Agilent 1260 series is used as a liquid chromatograph. A Thermo ODS-2 Hypersil C18 column (250 mm×4.0 mm) is used as a chromatographic column. A glucosamine salt sample to be tested is filtered with a 0.22 μm microfiltration membrane, and then injected into the chromatographic column at an injection volume of 10 μl. The N-acetylglucosamine and acetic acid are analyzed by an HPX-87H column (Bio-Rad, USA) as a chromatographic column. A differential refractometer detector is used. 5 mmol/L H₂SO₄ is used as a mobile phase, and detection is carried out at a flow rate of 0.6 ml/min and a temperature of 40° C.

### Example 1

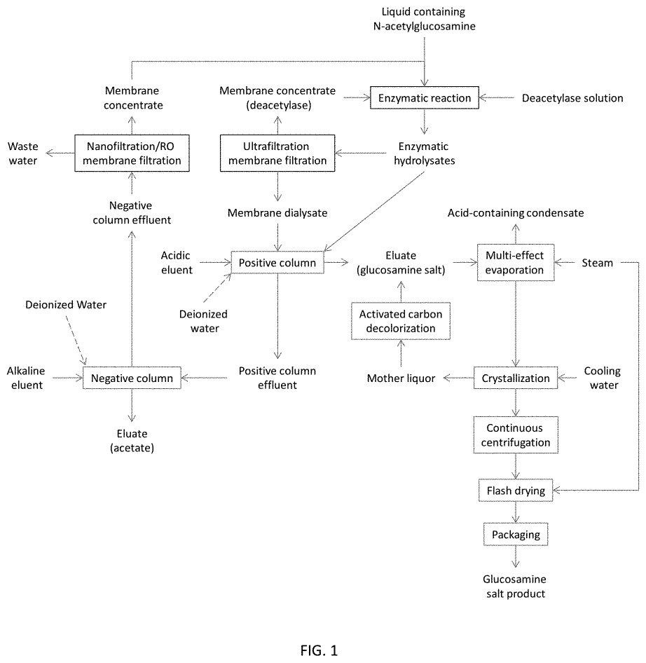

According to the process route as shown in FIG. 1, operation steps are as follows.

(1) An N-acetylglucosamine solution with a concentration of 40-150 g/L was used as a raw material, a deacetylase solution or a deacetylase preparation was added in a proportion of 10-30 U/g of N-acetylglucosamine, and an enzymatic reaction was carried out under stirring in a pH range of 4.0-8.0 at a temperature of 25-45° C. for 10-40 minutes. The deacetylase preparation was selected from commercial enzymes capable of removing acetyl in N-acetylglucosamine by hydrolysis. In the step, the acetyl in an N-acetylglucosamine molecule was removed specifically. An enzymatic hydrolysis product including glucosamine and acetic acid as main components was obtained after the reaction. The enzymatic reaction was preferably carried out at a pH of 7.0-8.0.

(2) The enzymatic hydrolysis product obtained after the reaction in step (1) was transported to an ultrafiltration membrane device. The ultrafiltration membrane had a molecular weight cut-off of 5-200 kDa, preferably 5-30 kDa. A membrane concentrate was subjected to dialysis with water, and an ultrafiltration membrane dialysate containing glucosamine and a membrane concentrate containing deacetylase were separately collected. After dialysis with a membrane was completed, the recovery rate of the deacetylase was 80-90%.

(3) An enzyme solution obtained after concentration with the membrane in step (2) was sent back to a deacetylase solution storage tank to participate in a next batch of enzymatic reaction process.

The ultrafiltration membrane dialysate containing glucosamine obtained in step (2) was continuously pumped into a positive column filled with an acidic resin (such as a cation exchange resin with a sulfonyl group) of a simulated moving bed at a feeding flow rate of 2.0-10.0 BV/h to make the glucosamine in the dialysate adsorbed to the positive column.

The positive column was washed out with deionized water to obtain a positive column effluent containing N-acetylglucosamine and acetic acid.

The positive column was subjected to continuous elution with hydrochloric acid, sulfuric acid, phosphoric acid, pyruvic acid, citric acid, and other acidic eluents with a concentration of 0.30-3.0 mol/L at a temperature of 20-70° C., preferably 25° C., to obtain a eluate containing a corresponding glucosamine salt.

### Example 2

According to the process route as shown in FIG. 1, operation steps are as follows.

(1) An N-acetylglucosamine solution with a concentration of 40-150 g/L was used as a raw material, a deacetylase solution or a deacetylase preparation was added in a proportion of 10-30 U/g of N-acetylglucosamine, and an enzymatic reaction was carried out under stirring in a pH range of 4.0-8.0 at a temperature of 25-45° C. for 10-40 minutes. The deacetylase preparation was selected from commercial enzymes capable of removing acetyl in N-acetylglucosamine by hydrolysis. In the step, the acetyl in an N-acetylglucosamine molecule was removed specifically. An enzymatic hydrolysis product including glucosamine and acetic acid as main components was obtained after the reaction. The enzymatic reaction was preferably carried out at a pH of 7.0-8.0.

(2) The enzymatic hydrolysis product obtained after the reaction in step (1) was transported to an ultrafiltration membrane device. The ultrafiltration membrane had a molecular weight cut-off of 5-200 kDa, preferably 5-30 kDa. A membrane concentrate was subjected to dialysis with water, and an ultrafiltration membrane dialysate containing glucosamine and a membrane concentrate containing deacetylase were separately collected. After dialysis with a membrane was completed, the recovery rate of the deacetylase was 80-90%.

(3) An enzyme solution obtained after concentration with the membrane in step (2) was sent back to a deacetylase solution storage tank to participate in a next batch of enzymatic reaction process.

The ultrafiltration membrane dialysate containing glucosamine obtained in step (2) was continuously pumped into a positive column filled with an acidic resin (such as a cation exchange resin with a sulfonyl group) of a simulated moving bed at a feeding flow rate of 2.0-10.0 BV/h to make the glucosamine in the dialysate adsorbed to the positive column.

The positive column was washed out with deionized water to obtain a positive column effluent containing N-acetylglucosamine and acetic acid.

The positive column was subjected to continuous elution with hydrochloric acid, sulfuric acid, phosphoric acid, pyruvic acid, citric acid, and other acidic eluents with a concentration of 0.30-3.0 mol/L at a temperature of 20-70° C., preferably 25° C., to obtain a eluate containing a corresponding glucosamine salt, which was used in subsequent processes of concentration, crystallization, and drying.

(4) The positive column effluent in step (3) was further transported to a negative column filled with an alkaline anion exchange resin (such as an anion exchange resin with a quaternary ammonium salt) at a feeding flow rate of 2.0-10.0 BV/h. Adsorption and elution were conducted at a temperature of 20-65° C., preferably 25° C., to make the acetic acid adsorbed to the negative column.

The negative column was washed out with deionized water to obtain a negative column effluent containing N-acetylglucosamine.

The negative column was subjected to continuous elution with NaOH or KOH and other alkaline eluents with a concentration of 0.30-3.0 mol/L at a rate of 1.0-8.0 BV/h to separate a eluate rich in acetate. The eluate was transported to a sewage treatment workshop to serve as a supplementary carbon source for nitrogen and phosphorus removal processes, and was also used as a chemical raw material.

(5) The negative column effluent flowing through the negative column in step (4) was transported to a nanofiltration membrane concentration device or a reverse osmosis membrane concentration device.

The nanofiltration membrane was a ceramic membrane with a pore size of 0.5-2 nm and an operation pressure of 2-5 atm.

The reverse osmosis membrane was an organic spiral-wound membrane or a ceramic membrane with a molecular weight cut-off of 50-100 Da and an operation pressure of 4-10 atm.

The N-acetylglucosamine obtained after concentration with the membrane had a final concentration of 10-15% (w/v). A membrane concentrate was recycled to an N-acetylglucosamine storage tank to participate in a next batch of enzymatic reaction process in step (1).

(6) The eluate of the positive column in step (3) was pumped into a multiple-effect evaporator for evaporation concentration. The multiple-effect evaporation concentration was triple-effect evaporation at gradient temperature. For example, the first-effect evaporator, the second-effect evaporator and the third-effect evaporator had a temperature of 80° C., 70° C. and 60° C. respectively, and the third-effect evaporator had a vacuum degree of 80-98 kPa.

(7) An effluent obtained after the concentration by the multiple-effect evaporator in step (6) flowed into a crystallizer. The crystallization temperature was controlled at 5-40° C. by controlling the temperature of a jacket of the crystallizer. A crystal suspension generated by the crystallizer was sent to a solid-liquid separation device for separation to obtain a mother liquor and a glucosamine salt crystal mud separately. A centrifuge with a continuous centrifugal function or a horizontal scraper discharge centrifuge was selected as the separation device.

(8) The mother liquor obtained after the separation in step (7) was subjected to decolorization with activated carbon. After the decolorization was completed, the mother liquor was sent back to the multiple-effect evaporator in step (6). The glucosamine salt crystal mud was transported to a drying device to obtain a dried glucosamine salt crystal. The recovery rate of the concentration, crystallization and drying units can reach 98%.

### Example 3

According to the process route as shown in FIG. 1, operation steps are as follows.

(1) 350 m³ of an N-acetylglucosamine solution with a concentration of 102 kg/m³ was collected in a storage tank and pumped into an enzymatic reaction tank, a deacetylase solution was added in a proportion of 10-25 U/g of N-acetylglucosamine, and an enzymatic reaction was carried out under stirring at a pH of 7.0-8.0 and a temperature of 37° C. for 30 minutes.

(2) An enzymatic hydrolysis product obtained after the reaction in step (1) was pumped into an ultrafiltration membrane device. The ultrafiltration membrane had a molecular weight cut-off of 5,000 Da. 60 m³ of pure water was added for dialysis of a membrane concentrate, and an ultrafiltration membrane dialysate and a membrane concentrate were separately collected. A glucosamine dialysate with a total content of 380 m³ and a concentration of 56.3 kg/m³ was collected. 30 m³ of an enzyme solution of the membrane concentrate was sent back to a deacetylase solution storage tank to participate in a next batch of enzymatic reaction process.

(3) The glucosamine dialysate obtained in step (2) was continuously pumped into a positive column filled with a 001×7 strongly acidic styrene resin of a simulated moving bed at a feeding flow rate of 4.0 BV/h. Feeding and elution were conducted at a temperature of 25° C. The glucosamine in the dialysate was adsorbed to the positive column. The positive column was washed out with deionized water to obtain a positive column effluent containing a neutral sugar and acetic acid.

The positive column was subjected to continuous elution with a hydrochloric acid solution with a concentration of 2 mol/L at a flow rate of 3.0 BV/h to obtain 91.7 m³ of a eluate containing a glucosamine hydrochloride with a concentration of 252 kg/m³.

(4) The positive column effluent in step (3) was further transported to a negative column filled with an anion exchange resin of the simulated moving bed at a flow rate of 4.0 BV/h. The negative column was washed out with deionized water to obtain a negative column effluent containing N-acetylglucosamine with a concentration of about 3%.

The negative column was subjected to continuous elution with a NaOH solution with a concentration of 1.5 mol/L to separate 80.6 m³ of an eluate rich in sodium acetate (107.5 kg/m³). The negative column was filled with a 201×7 strongly alkaline styrene resin.

(5) 514.6 m³ of the column effluent flowing through the negative column in step (4) was transported to a nanofiltration membrane concentration device. The nanofiltration membrane had a pore size of 1 nm and an operation pressure of 0.5-1.0 atm. A nanofiltration membrane concentrate containing N-acetylglucosamine with a total content of 89.3 m³ and a concentration of 125 g/L was obtained after concentration. The nanofiltration membrane concentrate was recycled to an N-acetylglucosamine storage tank to participate in a next batch of enzymatic reaction process.

In the first reaction and purification process, 23.1 tons of a glucosamine hydrochloride can be obtained, and the recovery rate of the glucosamine hydrochloride reaches 66.2%.

After the second circular reaction that 89.3 m³ of the concentrate obtained after concentration with the nanofiltration membrane was circulated to the N-acetylglucosamine solution storage tank and the enzyme solution of the membrane concentrate in step (2) was sent back to the enzymatic reaction tank, a glucosamine hydrochloride solution with a content of 45 m³ and a concentration of 238 kg/m³ can be recovered, and the total recovery rate of the glucosamine hydrochloride reaches 97%.

To continue the above process, eluents of the glucosamine hydrochloride obtained in step (3) in the first batch reaction and the second batch reaction were combined, and then subjected to concentration, crystallization, and drying. Details are as follows.

(6) A combined solution containing the glucosamine hydrochloride was pumped into a triple-effect evaporator at a feeding flow rate of 6 m³/h. A last-effect evaporator had a vacuum degree of 90 kPa. Cooling water had an inlet water temperature of 8-15° C., and a discharged product had a concentration of 720 g/L.

(7) An effluent of the triple-effect evaporator flowed into a crystallizer. The crystallization temperature was controlled at 40° C. by controlling the temperature of a jacket of the crystallizer. A crystal suspension produced by the crystallizer was sent to a horizontal scraper discharge centrifuge for separation to obtain a mother liquor and a glucosamine hydrochloride crystal mud separately.

(8) The mother liquor was sent to an activated carbon decolorization column at a flow rate of 0.5 m³/h for decolorization. After the decolorization was completed, the mother liquor was sent to the storage tank in front of the triple-effect evaporator. The glucosamine hydrochloride crystal mud separated by the horizontal scraper discharge centrifuge was sent to a flash dryer through a screw conveyor for flash drying at an inlet air temperature of 150° C. and an outlet air temperature of 80° C. to obtain a glucosamine hydrochloride crystal.

The total recovery rate of the concentration, crystallization and drying units in steps (6)-(8) can reach 98%.

In the whole production process, when 1 ton of the glucosamine hydrochloride is produced, 500 kg of a concentrated hydrochloric acid solution, 550 L of a 30% NaOH solution and 10 tons of pure water are consumed, 9.7 tons of wastewater is produced, and about 10 kg of the cation exchange resin and the anion exchange resin are separately consumed.

### Example 4

According to the process route as shown in FIG. 1, the difference is that on the basis of Example 3, a nanofiltration membrane concentration device was replaced with a reverse osmosis ceramic membrane device, and that is to say, a negative column effluent obtained in step (4) was collected into the reverse osmosis ceramic membrane device. The reverse osmosis membrane had a pore size of 1 nm and an operation pressure of 0.5-1.0 MPa. A liquid obtained after concentration by the reverse osmosis ceramic membrane device was sent back to an enzymatic reaction tank to participate in a next batch of enzymatic reaction process.

In the production process, when 1 ton of a glucosamine hydrochloride is produced, 11 tons of pure water is consumed, and 10.5 tons of wastewater is produced.

### Example 5

According to the process route as shown in FIG. 1, the difference is that on the basis of Example 3, a hydrochloric acid solution in step (3) was replaced with a sulfuric acid solution, and that is to say, in step (3), glucosamine adsorbed to a positive column was subjected to elution with 1 mol/L of the sulfuric acid solution to obtain a product, namely a glucosamine sulfate.

### Example 6

According to the process route as shown in FIG. 1, the difference is that on the basis of Example 3, a hydrochloric acid solution in step (3) was replaced with a phosphoric acid solution, and that is to say, in step (3), glucosamine adsorbed to a positive column was subjected to elution with 1 mol/L of the phosphoric acid solution to obtain a product, namely a glucosamine phosphate.

### Example 7

According to the process route as shown in FIG. 1, the difference is that on the basis of Example 3, a hydrochloric acid solution in step (3) was replaced with a citric acid solution, and that is to say, in step (3), glucosamine adsorbed to a positive column was subjected to elution with 1 mol/L of the citric acid solution to obtain a product, namely a glucosamine citrate.

### Example 8

According to the process route as shown in FIG. 1, the difference is that on the basis of Example 3, a hydrochloric acid solution in step (3) was replaced with a pyruvic acid solution, and that is to say, in step (3), glucosamine adsorbed to a positive column was subjected to elution with 2 mol/L of the pyruvic acid solution to obtain a product, namely a glucosamine pyruvate.

### Example 9

According to the process route as shown in FIG. 1, the difference is that on the basis of Example 3, a simulated moving bed in step (3) was replaced with a continuous moving bed device. A positive column and a negative column were filled with unchanged resins. The positive column was filled with a 001×7 strongly acidic styrene cation exchange resin, and the negative column was filled with a 201×7 strongly alkaline styrene anion exchange resin. When 1 ton of a product is produced, 16 kg of the cation exchange resin, 14 kg of the anion exchange resin, 600 kg of concentrated hydrochloric acid and 650 L of a 30% sodium hydroxide solution are consumed, and 15 tons of wastewater is produced.

### Example 10

According to the process route as shown in FIG. 1, the differences are that on the basis of Example 3, a simulated moving bed in step (3) was replaced with a fixed bed ion exchange device, a positive column was filled with a 201×7 strongly alkaline styrene anion exchange resin, and a negative column was filled with a 001×7 strongly acidic styrene cation exchange resin. When 1 ton of a product is produced, 30 kg of the cation exchange resin, 28 kg of the anion exchange resin, 1,300 kg of concentrated hydrochloric acid and 1,400 L of a 30% sodium hydroxide solution are consumed, and 50 tons of wastewater is produced.

### Example 11

According to the process route as shown in FIG. 1, the differences are that on the basis of Example 5, triple-effect evaporation concentration and crystallization were omitted, an eluate rich in a glucosamine sulfate obtained after elution in a positive column of a simulated moving bed was directly transported to a spray drying device at a feeding flow rate of 5 m³/h, and spray drying was conducted at an inlet air temperature of 150° C. After the drying was completed, 41.5 tons of a glucosamine sulfate powder was obtained. The purity of the product reaches 99%.

### Example 12

According to the process route as shown in FIG. 1, the differences are that on the basis of Example 3, an enzymatic reaction solution obtained in step (1) was continuously pumped into a positive column in step (3) at a feeding flow rate of 3.0 BV/h, feeding and elution were conducted at a temperature of 30° C., subsequent steps were the same as those in Example 2, and a product, namely a glucosamine hydrochloride, was obtained.

### Comparative Example 1

With reference to a method for separating a D-glucosamine hydrochloride in a patent application with an application number of CN2016112278411, 50 m³ of an N-acetylglucosamine solution with a concentration of 102 kg/m³ in a storage tank was collected, steam was introduced to heat the solution to 95° C., and the solution was pumped into an exchange column filled with a strongly acidic cation exchange resin at a temperature of 90° C. to make the N-acetylglucosamine undergo a reaction with the cation exchange resin for 240 minutes. After the reaction was completed, pure water was introduced to wash the cation exchange resin, and 40 m³ of a column effluent containing acetic acid was collected. After the washing was completed, 12% of hydrochloric acid was introduced to conduct elution on the cation exchange column, where the elution was conducted at a flow rate of 1.5 BV/h. 42 m³ of a eluate containing a glucosamine hydrochloride with a concentration of 103 kg/m³ was collected, and the recovery rate of this step was 88.6%. 0.2% (w/v) of activated carbon was added into the eluate for decolorization, and then the solution was pumped into a triple-effect evaporation concentrator, followed by crystallization, centrifugation, and drying to obtain 4,410 kg of a glucosamine hydrochloride with a total recovery rate of 80.2%. Since the reaction of the N-acetylglucosamine and the cation exchange resin is carried out at high temperature for a long time, a pigment is likely to be produced in the reaction process, and the loss of the ion exchange resin is increased. When a ton of the glucosamine hydrochloride is produced, 1,100 kg of concentrated hydrochloric acid and 120 kg of the cation exchange resin are consumed, 30 m³ of wastewater with a high content of an inorganic acid and high COD is produced, and the acetic acid cannot be recovered in the production process.

TABLE 1
Extraction effects of a glucosamine salt with different cation exchange modes

| | | | Use amount of | | |
|---|---|---|---|---|---|
| | | | a concentrated | | |
| | | | hydrochloric | Production of | |
| | Cation | Loss of resin | acid solution | wastewater | Product |
| Case | exchange | (kg/ton of | (kg/ton of | (m³/ton of | recovery rate |
| number | mode | product) | product) | product) | (%) |
| Example 3 | Simulated | 20 | 500 | 9.7 | 99 |
| | moving bed | | | | |
| Example 9 | Continuous | 30 | 600 | 15 | 99 |
| | moving bed | | | | |
| Example 10 | Fixed bed | 58 | 1,300 | 50 | 95 |
| Comparative | Deacetylation | 120 | 1,100 | 30 | 87.4 |
| Example 1 | reaction coupled | | | | |
| | with cation | | | | |
| | exchange resin | | | | |
| | adsorption | | | | |

The inventor has also tried to adjust process parameters of enzymatic hydrolysis, separation and purification to achieve the effects that within the range of the preferred parameters in Example 1, the product recovery rate of the ion exchange unit can reach 99%, the loss of the filling material is controlled within the range of 20 kg/ton, the use amount of the acidic solution is controlled within the range of 600 kg/ton of product, and the production of the wastewater is less than 10 m³/ton of product.

### Comparative Example 2

With reference to a method disclosed in CN2013106719979, the differences are that a raw material, namely a glucosamine hydrochloride mother liquor, was replaced with an enzymatic reaction solution containing glucosamine, and the operation step of transferring a eluate of a cation exchange column to an anion exchange column was omitted. A total of 50 m³ of a deacetylase reaction solution and a membrane dialysate (containing glucosamine and acetic acid) obtained after dialysis with an ultrafiltration membrane were collected in a storage tank. An N-acetylglucosamine solution with a concentration of 89 kg/m³ was pumped into an exchange column filled with a strongly acidic cation exchange resin at a temperature of 32° C. to make the N-acetylglucosamine adsorbed to the cation exchange resin. Pure water was introduced to wash out the cation exchange resin, and a total of 60 m³ of a column effluent containing acetic acid was collected. After the washing was completed, 0.3 mol/L of a hydrochloric acid solution was introduced to conduct elution on the cation exchange column, where the elution was conducted at a flow rate of 1.5 BV/h. 31 m³ of a glucosamine hydrochloride solution with a concentration of 126 kg/m³ was collected, and the recovery rate was 87.8%. A eluate was heated to 60° C., 1% of powdered activated carbon was added for decolorization, and after filtration was completed, 30.5 m³ of a glucosamine hydrochloride solution containing a filtrate with a concentration of 125 kg/m³ was obtained. Then, the solution was pumped into a triple-effect evaporation concentrator, followed by crystallization, centrifugation, and drying to obtain 3,585 kg of a light yellow glucosamine hydrochloride crystal with a total recovery rate of 80.6%. According to the method, since the decolorization and concentration are conducted in sequence, the consumption of the activated carbon is high. When a ton of the glucosamine hydrochloride is produced, 900 kg of concentrated hydrochloric acid and 20 kg of the cation exchange resin are consumed, and 30 m³ of wastewater with high COD is produced.

Although the present disclosure has been disclosed as preferred examples described above, the preferred examples are not intended to limit the present disclosure. Various changes and modifications can be made by any person familiar with the art without departing from the spirit and scope of the present disclosure. Therefore, the protection scope of the present disclosure shall be as defined by the claims.

1. A method for enzymatic production of a glucosamine salt and a purification method therefor, comprising:
subjecting a solution containing N-acetylglucosamine as a raw material to catalytic hydrolysis with deacetylase or a preparation containing deacetylase to obtain an enzymatic hydrolysis product;
subjecting the enzymatic hydrolysis product to filtration with a membrane, recovering the deacetylase from an obtained membrane retentate, recycling the deacetylase for the catalytic hydrolysis, and using an obtained membrane dialysate in cation exchange treatment;
subjecting the enzymatic hydrolysis product or the membrane dialysate to cation exchange and elution with an acidic eluent to obtain a glucosamine salt; and
subjecting a positive column effluent obtained in the cation exchange process to anion exchange and elution with an alkaline eluent to recover acetate, and subjecting a negative column effluent obtained in the anion exchange process to concentration and circular sending back to the catalytic hydrolysis process involving the deacetylase.

2. The method according to claim 1, wherein the membrane is a membrane assembly prepared from a ceramic material or a membrane assembly prepared from an organic material; an ultrafiltration membrane has a molecular weight cut-off of 5-200 kDa;
a resin for the cation exchange comprises a cation exchange resin with a sulfonyl group; a resin for the anion exchange comprises an anion exchange resin with a quaternary ammonium group; and
a device for the cation exchange and/or the anion exchange is a fixed bed for ion exchange, a continuous moving bed for ion exchange, or a simulated moving bed for ion exchange.

3. The method according to claim 1, wherein the acidic eluent is hydrochloric acid, sulfuric acid, phosphoric acid, pyruvic acid, or citric acid with a concentration of 0.3-4.0 mol/L.

4. The method according to claim 1, wherein the alkaline eluent is a NaOH solution or a KOH solution with a concentration of 0.30-3.0 mol/L.

5. The method according to claim 1, wherein in the ion exchange process, adsorption or elution is conducted at a temperature of 20-75° C. and a feeding flow rate of 2.0-10.0 BV/h; and the eluent has a flow rate of 1.0-8.0 BV/h.

6. The method according to claim 5, wherein the obtained glucosamine salt is subjected to concentration, crystallization, and drying to obtain a high-purity glucosamine salt.

7. The method according to claim 6, wherein the concentration is evaporation concentration;
the evaporation concentration is single-effect evaporation concentration or multiple-effect evaporation concentration, and a last-effect evaporator has a vacuum degree of 80-98 kPa;
the crystallization is conducted at a temperature of 5-40° C.;
the drying is low-temperature vacuum drying or flash drying; the low-temperature vacuum drying is conducted at a temperature of 40-80° C. and a vacuum degree of 70-95 kPa; and the flash drying is conducted at a hot air temperature of 120-300° C.

8. The method according to claim 7, wherein the method further comprises decolorization of a mother liquor; a decolorization method is adsorption decolorization with activated carbon; and a decolorized mother liquor is recycled in a concentration process.

9. The method according to claim 8, wherein according to the decolorization method, the consumption of the activated carbon is 0.01-2% (w/v) of that of a raw material solution; and the activated carbon is a carbon rod, a carbon column, or granular activated carbon.

10. The method according to claim 1, comprising the following steps:
(1) using an N-acetylglucosamine solution with a concentration of 40-150 g/L as a raw material, adding the preparation containing deacetylase, and carrying out an enzymatic reaction under stirring in a pH range of 4-8 at a temperature of 25-55° C. for 10-90 minutes;
(2) subjecting an enzymatic hydrolysis product obtained after the reaction in step (1) to filtration with an ultrafiltration membrane or a nanofiltration membrane to obtain an ultrafiltration membrane dialysate containing glucosamine and a membrane concentrate, and recycling an enzyme solution of the membrane concentrate to participate in a next batch of enzymatic reaction in step (1);
(3) subjecting the ultrafiltration membrane dialysate obtained in step (2) to adsorption with a cation exchange resin, subjecting the cation exchange resin to continuous elution with the acidic eluent to obtain an eluate containing a glucosamine salt, and washing out the cation exchange resin with deionized water to obtain the positive column effluent containing N-acetylglucosamine and acetic acid;
(4) subjecting the positive column effluent flowing through the cation exchange resin in step (3) to adsorption with an anion exchange resin, and subjecting the anion adsorption resin to elution with the alkaline eluent to separate an eluate containing acetate; and
(5) recycling the negative column effluent flowing through the anion exchange resin in step (4) to participate in a next batch of enzymatic reaction process in step (1), or subjecting the negative column effluent flowing through the anion exchange resin in step (4) to concentration first, followed by recycling to participate in a next enzymatic reaction process in step (1), wherein a concentration method is vacuum concentration, concentration by filtration with a nanofiltration membrane or a reverse osmosis membrane, or multiple-effect evaporation concentration.

11. The method according to claim 10, wherein in step (5), the nanofiltration membrane is a ceramic membrane with a pore size of 0.5-2 nm; and the reverse osmosis membrane is an organic spiral-wound membrane or a ceramic membrane.

12. The method according to claim 10, wherein the eluate containing the glucosamine salt obtained in step (3) is directly transported to a spray drying device at a feeding flow rate of 5 m³/h, and spray drying is conducted at an inlet air temperature of 150° C.
